(19) United States
(12) Patent Application Publication
(10) Pub. No.: US 2026/0102554 A1
(43) Pub. Date: Apr. 16, 2026

(54) SWABBABLE VALVE WITH UNIVERSAL PROXIMAL CONNECTOR

(71) Applicant: DARCO International Corporation, Sunrise, FL (US)

(72) Inventors: David A. RUBEN, Sunrise, FL (US); Alexander J. RUBEN, Sunrise, FL (US)

(21) Appl. No.: 19/357,219

(22) Filed: Oct. 14, 2025

Related U.S. Application Data
(60) Provisional application No. 63/707,971, filed on Oct. 16, 2024.

(51) Int. Cl. A61M 5/00; A61J 1/20; A61J 1/22

(52) U.S. Cl. CPC A61M 5/007; A61J 1/201; A61J 1/2041; A61J 1/2048; A61J 1/22

(57) ABSTRACT

A medical transfer set includes flexible tubing and a swabbable valve operatively coupled to an end of the flexible tubing, the swabbable valve including a body having opposing distal and proximal ends and defining an interior, a flexible plug arranged within the interior and providing an inner flow path extending between opposing distal and proximal ends of the flexible plug, and a universal proximal connector provided at the proximal end of the body and including a stem and a head extending from the stem. The universal proximal connector is configured to be interconnected and used with both a luer nut syringe and a luer lock syringe.

### BACKGROUND

A swabbable valve, also known as a needleless connector or swabbable cap, is a medical device used in intravenous (IV) therapy to provide a safe and efficient way to access a patient's IV line without the need for needles. In the context of a computed tomography (CT) procedure, a swabbable valve is particularly useful for administering contrast agents or medications intravenously. The contrast agent enhances the visibility of certain tissues and structures in the body, allowing for more accurate imaging. The swabbable valve ensures that the administration of the contrast agent is done safely, efficiently, and with minimal risk of infection or complications.

Once the swabbable valve is used and disconnected from a syringe, its valve face may be disinfected and cleaned (e.g., via swabbing with alcohol wipe, force it against a sponge filled with a sanitizing fluid, immersion in a sanitizing fluid, etc.) before being used with a new syringe. This advantageously allows the swabbable valve to be used multiple times before being disposed.

Proximal connectors for conventional swabbable valves are configured for a single type of syringe, but are unable to be operatively coupled to multiple syringe types. Consequently, users are required to purchase and implement multiple types of swabbable valves to be able to facilitate connection to a corresponding number and types of syringes.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 is a cross-sectional side view of an example transfer set that can be used in a computed tomography (CT) procedure, according to one or more embodiments of the present disclosure.

FIG. 2A illustrates an example luer nut syringe.

FIG. 2B illustrates an example luer lock syringe.

FIG. 3A is an enlarged view of the swabbable valve of FIG. 1 from the luer nut syringe of FIG. 2A, according to one or more embodiments.

FIG. 3B shows the swabbable valve of FIG. 1 operatively coupled to the luer nut syringe of FIG. 2A.

FIG. 4A is an enlarged view of the swabbable valve of FIG. 1 from the luer lock syringe of FIG. 2B, according to one or more embodiments.

FIG. 4B shows the swabbable valve of FIG. 1 operatively coupled to the luer lock syringe of FIG. 2B.

### DETAILED DESCRIPTION

The present disclosure is related to high pressure syringes and medical transfer sets and, more specifically, to a swabbable valve of a medical transfer set that includes a universal proximal connector capable of being operatively coupled to a plurality of high pressure syringes.

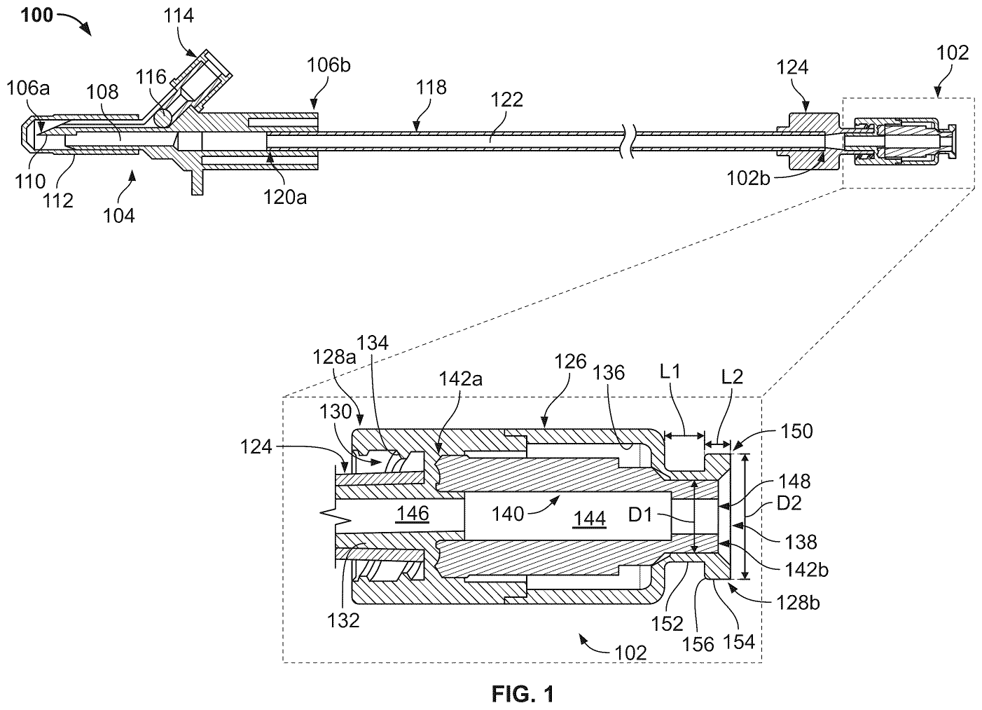

FIG. 1 is a cross-sectional side view of an example medical transfer set 100 that incorporates the principles of the present disclosure. The medical transfer set 100 is a sterile medical device that can be to safely and efficiently transfer fluids such as medications, IV solutions, contrast media, or blood components between different containers, syringes, or pumps. In at least one embodiment, the medical transfer set 100 can be used in a computed tomography (CT) procedure, and may be used in conjunction with a power injector (not shown) to safely and efficiently draw saline and/or a contrast media from multi-dose bulk containers; e.g., a saline IV bag, a rigid bottle of contrast agent, etc.

As described in more detail below, the medical transfer set 100 includes a swabbable valve 102, which allows the medical transfer set 100 to be used multiple times, and otherwise until the liquid volume within the bulk containers is depleted. This advantageously reduces setup time between patients and allows hospitals to use larger, more cost-effective fluid containers, which minimizes waste.

As illustrated, the medical transfer set 100 includes a piercing spike 104 that includes opposing first and second ends 106a and 106b, and a central channel 108 that extends between the first and second ends 106a, b. A sharpened tip or “spike” 110 is provided at the first end 106a and is sharpened to allow for smooth and easy insertion into an IV bag of saline or the rubber septum of a bulk bottle of contrast media without coring or causing significant resistance. To protect from inadvertent puncturing or harm to a user, the piercing spike 104 can include a sheath 112 that is removably attached to the piercing spike 104 to cover the spike 110 when not in use.

In some applications, the piercing spike 104 can include an air filter 114 that fluidly communicates with the central channel 108. The air filter 114 may include a check valve 116, such as a steel ball, or the like. The air filter 114 may help maintain proper fluid flow from an IV bag or a rigid container, such as a bulk bottle of contrast media, while simultaneously preventing airborne contaminants from entering the sterile solution.

The medical transfer set 100 further includes a length of flexible tubing 118 having opposing first and second ends 120a and 120b. The first end 120a may be operatively coupled to and extend from the second end 106b of the piercing spike 104. The tubing 118 is typically sterile and made of a flexible polymer. In some cases, the tubing 118 can be color-coded (e.g., clear for contrast media, blue for saline, etc.) to prevent mix-ups. The tubing 118 defines a lumen 122 that fluidly communicates with the central channel 108, thereby facilitating the transfer of fluids in either direction along the tubing 118.

The medical transfer set 100 further includes a connector 124 operatively coupled to the second end 120b of the tubing 118. The connector 124 provides a mechanical interface that attaches the swabbable valve 102 to the medical transfer set 100, thereby making the swabbable valve 102 an integral part of the medical transfer set 100.

As best seen in the enlarged image of FIG. 1, the swabbable valve 102 includes an elongate body 126 having a first or “distal” end 128a and a second or “proximal” end 128b. The connector 124 may be operatively coupled to the swabbable valve 102 at the distal end 128a. More specifically, the swabbable valve 102 may provide or otherwise define a connection cavity 130 at the distal end 128a, and an adapter 132 extends coaxially through the connection cavity 130. The connection cavity 130 may include threading 134 (e.g., luer threading), and an end of the connector 124 may be advanced into the connection cavity 130 and threaded to the swabbable valve 102 at the threading 134. As the connector 124 is threaded to the swabbable valve 102, the adapter 132 is advanced into an interior of the connector 124, which fluidly communicates with the lumen 122 of the tubing 118.

Once the two components are threaded together, the connector 124 and the swabbable valve 102 may be permanently (or immovably) secured to each other using an adhesive, for example. In other embodiments, the connector 124 may be removably attached to the swabbable valve 102, or operatively coupled to the swabbable valve 102 via other attachment means, such as laser welding at an interface between two components, an interference fit, a mechanical attachment (e.g., a J-slot), or any combination of the foregoing.

The body 126 may exhibit a generally circular and cylindrical geometry, and may define an interior 136. A valve opening 138 is defined at the proximal end 128b and facilitates access into the interior 136. In some embodiments, as illustrated, internal surfaces of the body at the valve opening 138 may be tapered or chamfered relative to a central axis of the body 126, thereby providing an angled entrance into the interior 136 of the body 126. The angled surfaces of the valve opening 138 may prove advantageous in helping to ease the insertion motion of the tip of a syringe, as will be described in more detail below.

A flexible diaphragm or “plug” 140 is arranged within the interior 136 and may be operable to stop and release the flow of a fluid (e.g., saline, a contrast agent, etc.) through the swabbable valve 102. In some embodiments, the flexible plug 140 may be made of silicone, but could alternatively be made of other flexible materials capable of providing or otherwise generating a sealed interface.

As illustrated, the flexible plug 140 provides opposing distal and proximal ends 142a and 142b, and an inner flow path 144 extends between the distal and proximal ends 142a, b. When the flexible plug 140 is properly received within the interior 136, the inner flow path 144 will align and fluidly communicate with an inner flow path 146 of the adapter 132, thereby facilitating fluid communication along the entire axial length of the swabbable valve 102, between the opposing distal and proximal ends 128a, b.

A plug opening 148 is provided at the proximal end 142b of the flexible plug 140 and provides access into the inner flow path 144. The plug opening 148 is provided at or near the valve opening 138 and provides access into the inner flow path 144. The flexible plug 140 is configured such that the plug opening 148 closes (seals) when a distal tip of a syringe is removed, thereby maintaining a sterile, closed system. In some embodiments, the proximal ends 128b, 142b of the body 126 and the flexible plug 140, respectively, may be aligned in the same plane. In such embodiments, the valve and plug openings 138, 148 may also be aligned with each other and otherwise flush. In other embodiments, however, and as shown in FIG. 1, the proximal end 142b of the flexible member 136 may be recessed within the body 126 a short distance, such that the proximal ends 128b, 142b are axially offset from each other and the valve and plug openings 138, 148 are misaligned with each other.

The swabbable valve 102 includes a universal proximal connector 150 provided at the proximal end 128b. According to embodiments of the present disclosure, the universal proximal connector 150 is designed and otherwise configured to allow the swabbable valve 102 to be interconnected and used with a plurality of types of syringes.

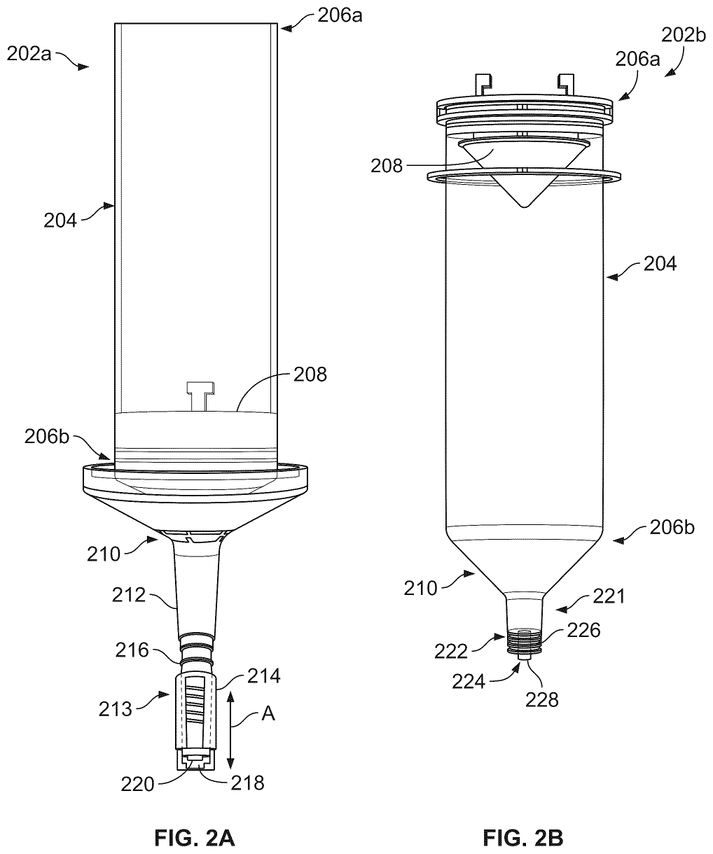

Referring to FIGS. 2A and 2B, illustrated are schematic side views of two types of syringes, shown as first syringe 202a and a second syringe 202b. As illustrated, each syringe 202a, b includes an elongate barrel 204 having opposing first and second ends 206a and 206b. A plunger 208 is slidably received within the interior of the barrel 204 and able to traverse the axial length of the barrel 204 between the first and second ends 206a, b. Each syringe 202a, b further includes or otherwise provides a hub 210 forming part of or extending from the second end 206b. The hub 210 provides a location where a proximal connector of a swabbable valve may be operatively and fluidly coupled to the corresponding syringe 202a, b, thereby enabling the transfer of fluids from the transfer set 100 (FIG. 1) to the barrel 204.

In FIG. 2A, the hub 210 includes a luer nut interface 213 configured to be operatively and fluidly coupled to a proximal connector of a swabbable valve, thereby placing the swabbable valve in fluid communication with the barrel 204 for the transfer of fluids. As illustrated, the luer nut interface 213 includes a pin member 212 and a helical luer nut 214 threadably and rotatably attached to the pin member 212. The pin member 212 defines threading 216, such as luer threading, and rotating the luer nut 214 relative to the pin member 212 will advance or retract the luer nut 214 along the axial length of the pin member 212 via the threading 216, as shown by the arrows A, and depending on the rotational direction.

The luer nut 214 also provides or defines a connector aperture 218 sized and otherwise configured to receive a corresponding proximal connector of a swabbable valve. Once the proximal connector of the swabbable valve is received within the connector aperture 218, rotating the luer nut 214 will correspondingly draw the swabbable valve toward or away from the pin member 212, depending on the rotational direction of the luer nut 214. To provide fluid communication between the barrel 204 and the swabbable valve, the luer nut 214 is rotated to draw the swabbable valve toward the barrel 204 and until a distal tip 220 of the pin member 212 is advanced into the interior of the swabbable valve.

Accordingly, the first syringe 202a with the luer nut interface 213 and the luer nut 214 will be referred to herein as the “luer nut”syringe 202a.

In FIG. 2B, the hub 210 of the second syringe 202b includes a luer lock interface 221 configured to be operatively and fluidly coupled to a proximal connector of a swabbable valve, thereby placing the swappable valve in fluid communication with the barrel 204. As illustrated, the luer lock interface 221 includes a luer lock 222 and a pin member 224 extends through and is concentric with the luer lock 222. The pin member 224 facilitates fluid communication with the interior of the barrel 204. As illustrated, threading 226 is defined on an inner surface of the luer lock 222, and the threading 226 may comprise luer threading. To provide fluid communication between the barrel 204 and a swabbable valve, a proximal connector of the swabbable valve is received within the interior of the luer lock 222, and the proximal connector is configured to be threaded to the threading 226. Once the proximal connector of the swabbable valve is received within the luer lock 222, rotating the swabbable valve relative to the second syringe 202b (or vice versa) will advance the swabbable valve toward the barrel 204 and until a distal tip 228 of the pin member 224 is advanced into the interior of the swabbable valve.

Accordingly, the second syringe 202b with the luer lock interface 221 and the luer lock 222 will be referred to herein as the “luer lock”syringe 202b.

Proximal connectors for conventional swabbable valve luers are designed for use with a single type of syringe, for example, either the luer nut syringe 202a or the luer lock syringe 202b, but the design prevents the conventional proximal connectors from being operatively coupled to both. Consequently, users are required to purchase and implement multiple types of swabbable valves with different proximal connector configurations to be able to facilitate connection to a corresponding number and types of syringes. According to embodiments of the present disclosure, the universal proximal connector 150 (FIG. 1) of the swabbable valve 102 (FIG. 1) is designed and otherwise configured to be interconnected and used with both the luer nut and luer lock syringes 202a, b.

Referring again to FIG. 1, as illustrated, the universal proximal connector 150 includes a stem 152 extending from or forming part of the body 126, and a head 154 extending from the stem 152. The head 154 provides and otherwise defines the “valve face” of the swabbable valve 102, and the valve face is configured to be “swabbed” with an antiseptic solution between syringe fillings. More specifically, the valve face may be cleaned and disinfected (e.g., via swabbing with an alcohol wipe, forcing it against a sponge filled with a sanitizing fluid, immersion in a sanitizing fluid, etc.) after being disconnected from a syringe and before interconnection with a new syringe, thus making the medical transfer set 100 multi-use. In some embodiments, the proximal end 142b of the flexible plug 140 can be flush with the valve face (e.g., the proximal end 128b of the body 126), thereby allowing the flexible plug 140 to be simultaneously “swabbed”and disinfected.

The stem 152 exhibits a first diameter D1, and the head 154 exhibits a second diameter D2 larger than the first diameter D1. Moreover, the stem 152 exhibits a first length L1 and the head 154 exhibits a second length L2. In some embodiments, the first length L1 is greater than the second length L2, and the ratio between L1 and L2 is a function of the thread pitch required to properly engage the universal proximal connector 150 to a corresponding syringe (e.g., either of the luer nut and luer lock syringes 202a, b). In other embodiments, however, the first length L1 may be smaller than the second length L2, or otherwise the first and second lengths L1, L2 may be substantially the same, without departing from the scope of the disclosure.

In at least one embodiment, as illustrated, the head 154 may provide a rounded or arcuate distal surface 156. The rounded geometry of the distal surface 156 may help the head 154 be received within a corresponding connector of the syringe, for example, the connector aperture 218 (FIG. 2A) of the luer nut interface 213 (FIG. 2A).

The geometry and dimensions D1, D2, L1, L2 of the universal proximal connector 150 makes the universal proximal connector 150 compatible with both the luer nut and luer lock syringes 202a, b (FIGS. 2A-2B) at the luer nut and luer lock interfaces 213, 222 (FIGS. 2A-2B), respectively. More specifically, the dimensions and geometry of the stem 152 and the head 154 may be such that the stem 152 and the head 154 are able to be received within the connector aperture 218 (FIG. 2A) and also within the interior of the luer lock 222 (FIG. 2B), which allows the swabbable valve 102 to be operatively coupled to both types of syringes 202a, b. As a result, a user is not required to acquire and implement two types of swabbable valve luers to be used with each syringe 202a, b type.

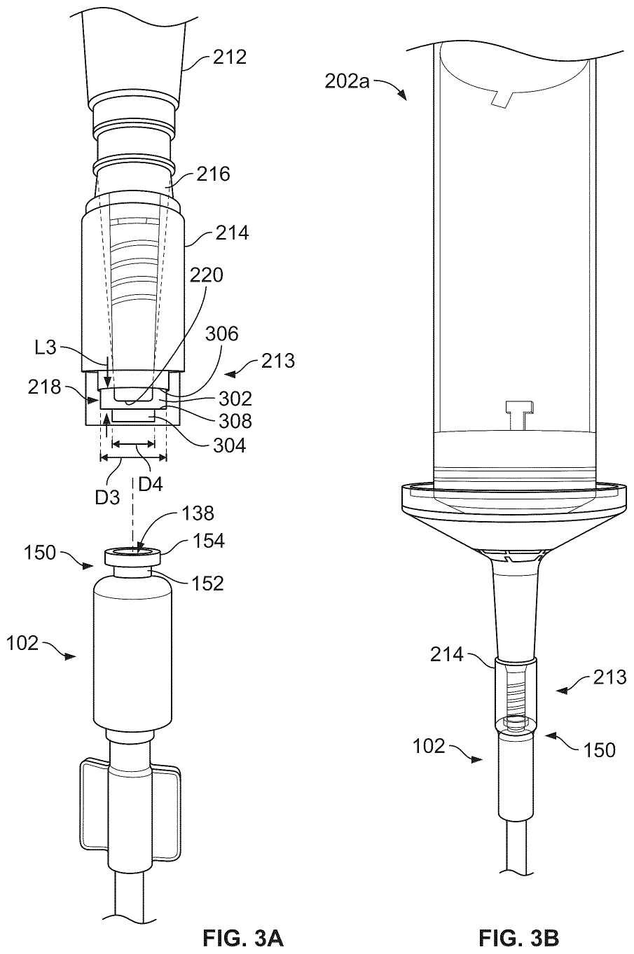

FIG. 3A is an enlarged view of the swabbable valve 102 disconnected from the distal end of the luer nut syringe 202a, and FIG. 3B shows the swabbable valve 102 operatively coupled to the luer nut syringe 202a, according to one or more embodiments. More specifically, FIG. 3A is an enlarged view of the universal proximal connector 150 of the swabbable valve 102 exploded from the luer nut interface 213, and FIG. 3B shows the universal proximal connector 150 operatively coupled to the luer nut interface 213.

Referring to FIG. 3A, the luer nut 214 defines the connector aperture 218, which includes a first or “head” aperture 302 and a second or “stem” aperture 304 that is contiguous with the head aperture 302. The head aperture 302 is sized and otherwise configured to receive the head 154 of the swabbable valve 102, and the stem aperture 304 is sized to receive a portion of the stem 152.

The head aperture 302 exhibits a third diameter D3 and the stem aperture 304 exhibit a fourth diameter D4, where the fourth diameter D4 is smaller than the third diameter D3. In at least one embodiment, the diameter D3 of the head aperture 302 may be slightly smaller than the diameter D2 (FIG. 1) of the head 154. In such embodiments, receiving the head 154 within the head aperture 302 may result in a snap fit or interference engagement. In other embodiments, however, the diameter D3 of the head aperture 302 may be larger than the diameter D2, and the head 154 may be received within the head aperture 302 with little or no interference or obstruction. Similarly, in at least one embodiment the fourth diameter D4 of the stem aperture 304 may be slightly smaller than the diameter D1 (FIG. 1) of the stem 152. In such embodiments receiving the stem 152 within the stem aperture 304 may result in a snap fit or interference engagement. In other embodiments, however, the fourth diameter D4 may be larger than the diameter D1, and the stem 152 may be received within the stem aperture 304 with little or no interference or obstruction.

The head aperture 302 provides and otherwise defines a proximal surface 306 and a radial shoulder 308 axially offset from the proximal surface 306. The head aperture 302 transitions to the stem aperture 304 at the radial shoulder 308. The head 154 is laterally received into the head aperture 302, and when the head 154 is received within the head aperture 302, the proximal end 128b (FIG. 1) of the swabbable valve 102 (e.g., the valve face) is engageable against the proximal surface, while the bottom (backside) of the head 154 rests against the radial shoulder 308. The radial shoulder 308 prevents the head 154, and thus the swabbable valve 102, from disconnecting from the luer nut 214 in the axial direction. Rather, the head 154 must be transitioned laterally out of the head aperture 302 to disconnect the swabbable valve 102 from the luer nut 214.

The axial distance between the proximal surface 306 and the radial shoulder 308 constitutes a third length L3, and in some embodiments, the third length L3 may be slightly smaller than the second length L2 of the head 154 (FIG. 1). In such embodiments, receiving the head 154 within the head aperture 302 may require a small amount of force and otherwise result in an interference fit between the two components parts. In other embodiments, however, the third length L3 may be slightly larger than the second length L2, and receiving the head 154 laterally within the head aperture 302 may occur with little or no interference or obstruction, without departing from the scope of the disclosure.

The universal proximal connector 150 is laterally received within the connector aperture 218. More specifically, the head 154 and the stem 152 may be simultaneously received laterally within the head and stem apertures 302, 304, respectively. Once the universal proximal connector 150 is received within the connector aperture 218, rotating the luer nut 214 will cause the luer nut 214 interact with the luer threading 216, and thereby advance the luer nut 214 along the length of the pin member 212. Advancing the luer nut 214 proximally will correspondingly force the head 154 into axial engagement with the radial shoulder 308, thereby drawing the swabbable valve 120 toward the pin member 212. Continued rotation of the luer nut 214 will cause the luer nut 214 to advance proximally along the axial length of the pin member 212, and the distal tip 220 of the pin member 212 will eventually advance into the interior of the swabbable valve 102 and, more specifically, through the valve opening 138 provided at the head 154. Advancing the luer nut 214 further in the proximal direction will eventually advance the distal tip 220 into the inner flow path 144 (FIG. 1) of the flexible plug 140 (FIG. 1). Once the distal tip 220 is located within the inner flow path 144, fluid communication between the luer nut syringe 202a and the swabbable valve 102 will be enabled.

The swabbable valve 102 may be disconnected from the luer nut syringe 202a by reversing the foregoing steps.

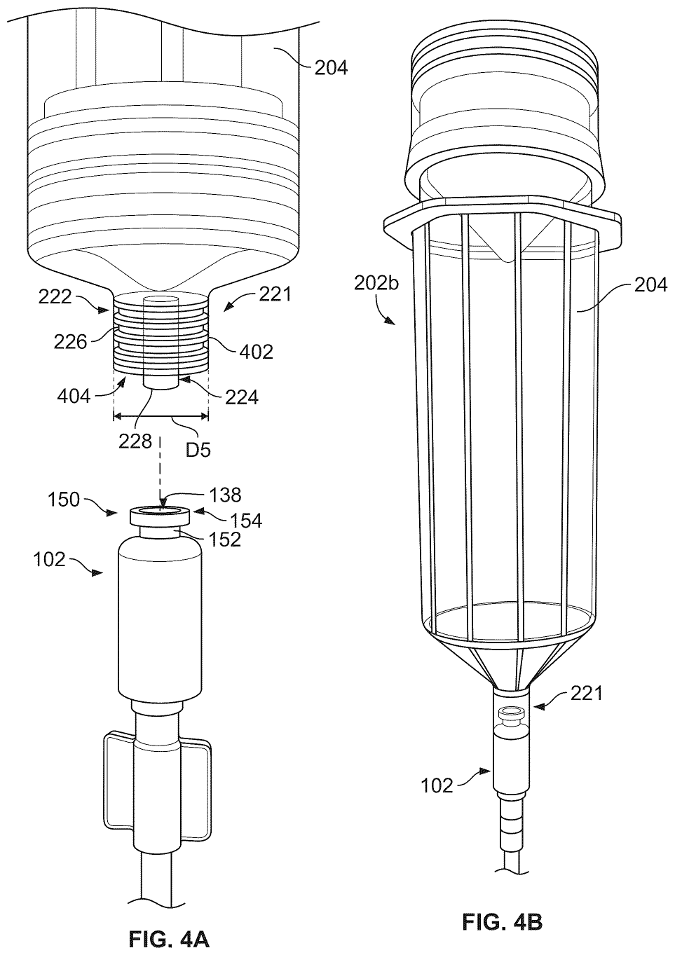

FIG. 4A is an enlarged view of the swabbable valve 102 disconnected from the distal end of the luer lock syringe 202b, and FIG. 4B shows the swabbable valve 102 operatively coupled to the luer lock syringe 202b, according to one or more embodiments. More specifically, FIG. 4A is an enlarged view of the universal proximal connector 150 of the swabbable valve 102 exploded from the luer lock interface 221, and FIG. 4B shows the universal proximal connector 150 operatively coupled to the luer lock interface 221.

Referring to FIG. 4A, the luer lock interface 221 includes the luer lock 222, which provides a cylindrical sidewall 402 that defines a lock interior 404. The pin member 224 extends within the lock interior 404 and is concentric with the cylindrical sidewall 402. In at least one embodiment, as illustrated, the distal tip 228 of the pin member 224 extends past a distal end of the luer lock 222.

The lock interior 404 may be sized to receive the universal proximal connector 150. More specifically, the cylindrical sidewall 402 may exhibit a fifth diameter D5, which is larger than the second diameter D2 (FIG. 1) of the head 154. Consequently, the head 154 may be able to be received within and advanced into the lock interior 404. The threading 226 is defined on an inner surface of the lock interior 404, and the head 154 may be sized and otherwise configured to be threaded into the threading 226. Accordingly, to provide fluid communication between the barrel 204 and the swabbable valve 102, the universal proximal connector 150 is received within the lock interior 404, and the head 154 is threaded into the threading 226 by rotating the swabbable valve 102 relative to the luer lock syringe 202b, or vice versa. Receiving the head 154 within the lock interior 404 may simultaneously advance the distal tip 224 of the pin member 222 into the interior of the swabbable valve 102 and, more specifically, through the valve opening 138 provided at the head 154. Rotating the swabbable valve 102 relative to the second syringe 202b (or vice versa) will thread the head 154 into the threading 226 and thereby advance the swabbable valve 120 proximally toward the barrel 204. Advancing the swabbable valve 102 further in the proximal direction will eventually advance the distal tip 224 into the inner flow path 144 (FIG. 1) of the flexible plug 140 (FIG. 1), and once the distal tip 224 is located within the inner flow path 144, fluid communication between the luer nut syringe 202a and the swabbable valve 102 will be enabled.

Embodiments disclosed herein include:
A. A medical transfer set includes a piercing spike, flexible tubing having opposing first and second ends, the piercing spike being operatively coupled to the first end, and a swabbable valve operatively coupled to the second end. The swabbable valve including a body having opposing distal and proximal ends and defining an interior, a flexible plug arranged within the interior and providing an inner flow path extending between opposing distal and proximal ends of the flexible plug, and a universal proximal connector provided at the proximal end of the body and including a stem and a head extending from the stem. Wherein the universal proximal connector is configured to be interconnected and used with both a luer nut syringe having a luer nut interface providing a first pin member and a luer nut threadably and rotatably attached to the first pin member, the luer nut defining a connector aperture sized to laterally receive the universal proximal connector, wherein, when the head and the stem are received within the connector aperture, rotating the luer nut draws the swabbable valve toward the first pin member to advance a distal tip of the first pin member into the inner flow path, and a luer lock syringe having a luer lock interface providing a luer lock and a second pin member extending within and concentric with the luer lock, wherein threading is defined on an inner surface of the luer lock, and the head is sized to be received within the luer lock and engageable with the threading such that rotating the swabbable valve relative to the luer lock syringe advances a distal tip of the second pin member into the inner flow path.
B. A swabbable valve for a medical transfer set includes a body having opposing distal and proximal ends and defining an interior, a flexible plug arranged within the interior and providing an inner flow path extending between opposing distal and proximal ends of the flexible plug, and a universal proximal connector provided at the proximal end of the body and including a stem and a head extending from the stem. The universal proximal connector is configured to be interconnected and used with both a luer nut syringe having a luer nut interface providing a first pin member and a luer nut threadably and rotatably attached to the first pin member, the luer nut defining a connector aperture sized to laterally receive the universal proximal connector, wherein, when the head and the stem are received within the connector aperture, rotating the luer nut draws the swabbable valve toward the first pin member to advance a distal tip of the first pin member into the inner flow path, and a luer lock syringe having a luer lock interface providing a luer lock and a second pin member extending within and concentric with the luer lock, wherein threading is defined on an inner surface of the luer lock, and the head is sized to be received within the luer lock and engageable with the threading such that rotating the swabbable valve relative to the luer lock syringe advances a distal tip of the second pin member into the inner flow path.
C. A method of transitioning a medical transfer set between syringes includes positioning the medical transfer set adjacent a luer nut syringe, the medical transfer set including a swabbable valve operatively coupled to flexible tubing and including a body having opposing distal and proximal ends and defining an interior, a flexible plug arranged within the interior and providing an inner flow path extending between opposing distal and proximal ends of the flexible plug, and a universal proximal connector provided at the proximal end of the body and including a stem and a head extending from the stem. The method further including operatively coupling the medical transfer set to a luer nut interface of the luer nut syringe, the luer nut interface including a first pin member and a luer nut threadably and rotatably attached to the first pin member, wherein operatively coupling the medical transfer set to the luer nut interface includes laterally receiving the head and the stem within the connector aperture, and rotating the luer nut and thereby drawing the swabbable valve toward the first pin member and advancing a distal tip of the first pin member into the inner flow path. The method further including disconnecting the medical transfer set from the luer nut syringe, operatively coupling the medical transfer set to a luer lock interface of a luer lock syringe, the luer lock interface providing a luer lock and a second pin member extending within and concentric with the luer lock, wherein operatively coupling the medical transfer set to the luer lock interface includes receiving the head within the luer lock and engaging the head with threading defined on an inner surface of the luer lock, and rotating the swabbable valve relative to the luer lock syringe and thereby advancing a distal tip of the second pin member into the inner flow path.

Each of embodiments A, B, and C may have one or more of the following additional elements in any combination: Element 1: wherein a valve opening is defined at the proximal end of the body, and a plug opening is defined at the proximal end of the flexible plug and positioned adjacent the plug opening, and wherein the plug opening provides access into the inner flow path. Element 2: wherein the connector aperture provides a head aperture sized to receive the head, and a stem aperture contiguous with the head aperture and sized to receive at least a portion of the stem. Element 3: wherein at least one of the head and the stem are received within the head aperture and the stem aperture via a snap fit engagement. Element 4: wherein the connector aperture further provides a radial shoulder that provides a transition between the head aperture and the stem aperture, and wherein a bottom of the head is engageable with the radial shoulder when the head is received within the head aperture. Element 5: wherein the luer lock provides a cylindrical sidewall that defines a lock interior, and the threading is defined on an inner wall of the cylindrical sidewall, and wherein the second pin member extends within the lock interior and is concentric with the cylindrical sidewall. Element 6: wherein the lock interior is sized to receive the head, and the head threadably engages the threading to advance the distal tip of the second pin member into the inner flow path of the flexible plug.

Element 7: wherein a valve opening is defined at the proximal end of the body, and a plug opening is defined at the proximal end of the flexible plug and positioned adjacent the plug opening, and wherein the plug opening provides access into the inner flow path. Element 8: wherein an internal surface of the body at the valve opening is angled. Element 9: wherein the plug opening closes when the distal tip of the first or second pin members is removed from the plug opening. Element 10: wherein the proximal end of the flexible member is recessed within the body and axially offset from the proximal end of the body. Element 11: wherein the stem exhibits a first diameter, and the head exhibits a second diameter larger than the first diameter. Element 12: wherein the connector aperture provides a head aperture sized to receive the head, and a stem aperture contiguous with the head aperture and sized to receive at least a portion of the stem. Element 13: wherein the connector aperture further provides a radial shoulder that provides a transition between the head aperture and the stem aperture, and wherein a bottom of the head is engageable with the radial shoulder when the head is received within the head aperture. Element 14: wherein the luer lock provides a cylindrical sidewall that defines a lock interior, and the threading is defined on an inner wall of the cylindrical sidewall, and wherein the second pin member extends within the lock interior and is concentric with the cylindrical sidewall. Element 15: wherein the lock interior is sized to receive the head, and the head is configured to be threaded into the threading to advance the distal tip of the second pin member into the inner flow path of the flexible plug.

Element 16: wherein the connector aperture provides a head aperture sized to receive the head, and a stem aperture contiguous with the head aperture and sized to receive at least a portion of the stem, the method further comprising receiving at least one of the head and the stem within the head aperture and the stem aperture via a snap fit engagement. Element 17: further comprising disconnecting the medical transfer set from the luer lock syringe, and closing a plug opening provided at a proximal end of the flexible plug upon disconnecting the luer nut syringe or the luer lock syringe.

By way of non-limiting example, exemplary combinations applicable to A, B, and C include: Element 2 with Element 3; Element 2 with Element 4; Element 5 with Element 6; Element 7 with Element 8; Element 7 with Element 9; Element 12 with Element 13; and Element 14 with Element 15.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The use of directional terms such as above, below, upper, lower, upward, downward, left, right, and the like are used in relation to the illustrative embodiments as they are depicted in the figures, the upward direction being toward the top of the corresponding figure and the downward direction being toward the bottom of the corresponding figure.

What is claimed is:

1. A medical transfer set, comprising:
a piercing spike;
flexible tubing having opposing first and second ends, the piercing spike being operatively coupled to the first end; and
a swabbable valve operatively coupled to the second end and including:
a body having opposing distal and proximal ends and defining an interior;
a flexible plug arranged within the interior and providing an inner flow path extending between opposing distal and proximal ends of the flexible plug; and
a universal proximal connector provided at the proximal end of the body and including a stem and a head extending from the stem;
wherein the universal proximal connector is configured to be interconnected and used with both:
a luer nut syringe having a luer nut interface providing a first pin member and a luer nut threadably and rotatably attached to the first pin member, the luer nut defining a connector aperture sized to laterally receive the universal proximal connector, wherein, when the head and the stem are received within the connector aperture, rotating the luer nut draws the swabbable valve toward the first pin member to advance a distal tip of the first pin member into the inner flow path; and
a luer lock syringe having a luer lock interface providing a luer lock and a second pin member extending within and concentric with the luer lock, wherein threading is defined on an inner surface of the luer lock, and the head is sized to be received within the luer lock and engageable with the threading such that rotating the swabbable valve relative to the luer lock syringe advances a distal tip of the second pin member into the inner flow path.

2. The medical transfer set of claim 1, wherein a valve opening is defined at the proximal end of the body, and a plug opening is defined at the proximal end of the flexible plug and positioned adjacent the plug opening, and wherein the plug opening provides access into the inner flow path.

3. The medical transfer set of claim 1, wherein the connector aperture provides:
a head aperture sized to receive the head; and
a stem aperture contiguous with the head aperture and sized to receive at least a portion of the stem.

4. The medical transfer set of claim 3, wherein at least one of the head and the stem are received within the head aperture and the stem aperture via a snap fit engagement.

5. The medical transfer set of claim 3, wherein the connector aperture further provides a radial shoulder that provides a transition between the head aperture and the stem aperture, and wherein a bottom of the head is engageable with the radial shoulder when the head is received within the head aperture.

6. The medical transfer set of claim 1, wherein the luer lock provides a cylindrical sidewall that defines a lock interior, and the threading is defined on an inner wall of the cylindrical sidewall, and wherein the second pin member extends within the lock interior and is concentric with the cylindrical sidewall.

7. The medical transfer set of claim 6, wherein the lock interior is sized to receive the head, and the head threadably engages the threading to advance the distal tip of the second pin member into the inner flow path of the flexible plug.

8. A swabbable valve for a medical transfer set, comprising:
a body having opposing distal and proximal ends and defining an interior;
a flexible plug arranged within the interior and providing an inner flow path extending between opposing distal and proximal ends of the flexible plug; and
a universal proximal connector provided at the proximal end of the body and including a stem and a head extending from the stem,
wherein the universal proximal connector is configured to be interconnected and used with both:
a luer nut syringe having a luer nut interface providing a first pin member and a luer nut threadably and rotatably attached to the first pin member, the luer nut defining a connector aperture sized to laterally receive the universal proximal connector, wherein, when the head and the stem are received within the connector aperture, rotating the luer nut draws the swabbable valve toward the first pin member to advance a distal tip of the first pin member into the inner flow path; and
a luer lock syringe having a luer lock interface providing a luer lock and a second pin member extending within and concentric with the luer lock, wherein threading is defined on an inner surface of the luer lock, and the head is sized to be received within the luer lock and engageable with the threading such that rotating the swabbable valve relative to the luer lock syringe advances a distal tip of the second pin member into the inner flow path.

9. The swabbable valve of claim 8, wherein a valve opening is defined at the proximal end of the body, and a plug opening is defined at the proximal end of the flexible plug and positioned adjacent the plug opening, and wherein the plug opening provides access into the inner flow path.

10. The swabbable valve of claim 9, wherein an internal surface of the body at the valve opening is angled.

11. The swabbable valve of claim 9, wherein the plug opening closes when the distal tip of the first or second pin members is removed from the plug opening.

12. The swabbable valve of claim 8, wherein the proximal end of the flexible member is recessed within the body and axially offset from the proximal end of the body.

13. The swabbable valve of claim 8, wherein the stem exhibits a first diameter, and the head exhibits a second diameter larger than the first diameter.

14. The swabbable valve of claim 8, wherein the connector aperture provides:
a head aperture sized to receive the head; and
a stem aperture contiguous with the head aperture and sized to receive at least a portion of the stem.

15. The swabbable valve of claim 14, wherein the connector aperture further provides a radial shoulder that provides a transition between the head aperture and the stem aperture, and wherein a bottom of the head is engageable with the radial shoulder when the head is received within the head aperture.

16. The swabbable valve of claim 8, wherein the luer lock provides a cylindrical sidewall that defines a lock interior, and the threading is defined on an inner wall of the cylindrical sidewall, and wherein the second pin member extends within the lock interior and is concentric with the cylindrical sidewall.

17. The swabbable valve of claim 16, wherein the lock interior is sized to receive the head, and the head is configured to be threaded into the threading to advance the distal tip of the second pin member into the inner flow path of the flexible plug.

18. A method of transitioning a medical transfer set between syringes, comprising:
positioning the medical transfer set adjacent a luer nut syringe, the medical transfer set including a swabbable valve operatively coupled to flexible tubing and including:
a body having opposing distal and proximal ends and defining an interior;
a flexible plug arranged within the interior and providing an inner flow path extending between opposing distal and proximal ends of the flexible plug; and
a universal proximal connector provided at the proximal end of the body and including a stem and a head extending from the stem;
operatively coupling the medical transfer set to a luer nut interface of the luer nut syringe, the luer nut interface including a first pin member and a luer nut threadably and rotatably attached to the first pin member, wherein operatively coupling the medical transfer set to the luer nut interface includes:
laterally receiving the head and the stem within the connector aperture; and
rotating the luer nut and thereby drawing the swabbable valve toward the first pin member and advancing a distal tip of the first pin member into the inner flow path;
disconnecting the medical transfer set from the luer nut syringe;
operatively coupling the medical transfer set to a luer lock interface of a luer lock syringe, the luer lock interface providing a luer lock and a second pin member extending within and concentric with the luer lock, wherein operatively coupling the medical transfer set to the luer lock interface includes:
receiving the head within the luer lock and engaging the head with threading defined on an inner surface of the luer lock; and
rotating the swabbable valve relative to the luer lock syringe and thereby advancing a distal tip of the second pin member into the inner flow path.

19. The method of claim 18, wherein the connector aperture provides a head aperture sized to receive the head, and a stem aperture contiguous with the head aperture and sized to receive at least a portion of the stem, the method further comprising receiving at least one of the head and the stem within the head aperture and the stem aperture via a snap fit engagement.

20. The method of claim 18, further comprising:
disconnecting the medical transfer set from the luer lock syringe; and
closing a plug opening provided at a proximal end of the flexible plug upon disconnecting the luer nut syringe or the luer lock syringe.
